(19) United States
(12) Patent Application Publication
(10) Pub. No.: US 2024/0044883 A1
(43) Pub. Date: Feb. 8, 2024

(54) DETECTION OF AIRBORNE ANALYTES USING IMPRINTED MICELLES

(71) Applicant: Battelle Memorial Institute, Richland, WA (US)

(72) Inventors: Samuel S. Morrison, Richland, WA (US); Lance R. Hubbard, Richland, WA (US); Caleb J. Allen, Richland, WA (US); Amy C. Sims, Richland, WA (US); Matthew J. O'Hara, Richland, WA (US); Nicolas E. Uhnak, Richland, WA (US)

(73) Assignee: Battelle Memorial Institute, Richland, WA (US)

(21) Appl. No.: 18/359,300

(22) Filed: Jul. 26, 2023

Related U.S. Application Data
(60) Provisional application No. 63/392,780, filed on Jul. 27, 2022.

(51) Int. Cl. G01N 33/543; G01N 33/569; G01N 27/413; G01N 27/28

(52) U.S. Cl. CPC G01N 33/54313; G01N 33/56983; G01N 27/413; G01N 27/283; G01N 2333/165

(57) ABSTRACT

Methods for the near real-time detection of airborne analytes using imprinted micelles and electrochemical cells are described. The methods demonstrate selectivity to the imprinted micelles over others that are of similar size and configuration and are compatible with airborne aerosol sampling techniques. The detection method can be used to monitor and detect any airborne analyte, including pathogens (such as SARS-CoV-2), toxins, proteins, organic molecules, inorganic particles, chemicals, explosive particles, and environmental pollutants.

### CROSS REFERENCE TO RELATED APPLICATIONS

This application claims the benefit of U.S. Provisional Application No. 63/392,780, filed Jul. 27, 2022, which is herein incorporated by reference in its entirety.

### ACKNOWLEDGMENT OF GOVERNMENT SUPPORT

This invention was made with Government support under Contract DE-AC05-76RL01830 awarded by the U.S. Department of Energy. The Government has certain rights in the invention.

### FIELD

This disclosure concerns methods for detection of airborne analytes, such as viruses, using imprinted micelles and electrochemical detection. In some examples, the methods allow for real-time detection using an ambient air sample.

### BACKGROUND

Real-time detection of airborne pathogens is desirable for controlling pandemics. The ability to detect pathogens, such as severe acute respiratory syndrome coronavirus 2 (SARS-CoV-2), in near real time would allow rapid screening and early detection of virus spread. Established detection methods for SARS-CoV-2 include rapid antigen tests, serological surveys, and reverse transcription-polymerase chain reaction. However, these methods are not currently compatible with airborne sampling (Kumar et al., Chem. Eng. J. 430(3):132966, 2022). Thus, a need exists for methods that enable detection of SARS-CoV-2 in ambient air samples, for example in real-time.

### SUMMARY

Detection of airborne particles of interest, for example in real-time, is needed for avoiding current and future pathogenic threats. As disclosed herein, the incorporation of imprinted particles into a micelle-based electrochemical cell produced a signal when brought into contact with particle analytes (such as SARS-CoV-2) previously imprinted onto the structure. Nanoamp scales of signals were generated from single virus-micelle interactions. The system described herein exhibits selectivity to the imprinted particles over others that are of similar size and configuration and is compatible with airborne aerosol sampling techniques. Thus, the application of imprinted micelle technology can provide near real-time detection methods to a multitude of analytes of interest.

Provided herein are methods of detecting an airborne analyte. The methods include contacting an ambient air sample suspected of containing the analyte with an aqueous aerosol and subjecting the air sample and aqueous aerosol to condensation, thereby producing a liquid-analyte solution; contacting the liquid-analyte solution with an imprinted micelle, wherein the imprinted micelle includes at least one analyte imprint (e.g., an imprint generated by the analyte) and contains an electrolyte solution; and detecting an electrochemical signal produced upon binding of the analyte to the imprinted micelle, releasing the electrolyte solution. In some aspects, the analyte is a pathogen, a toxin, a protein, an organic molecule, an inorganic particle, a chemical, an explosive particle, or an environmental pollutant. In some examples, the pathogen is a virus, such as SARS-CoV-2.

In some aspects of the disclosed methods, the liquid-analyte solution and imprinted micelle are contacted in an electrochemical cell. In some examples, in the electrochemical cell, the imprinted micelle is suspended in the aqueous solution. In other examples, in the electrochemical cell, the imprinted micelle is adhered to a solid support.

In some aspects, the method is a multiplex method and the imprinted micelle includes imprinted micelles specific for a plurality of analytes. In some examples, each imprinted micelle includes at least one molecularly imprinted particle (such as one that includes polymer, glass and/or metal) specific for each analyte. In other examples, each imprinted micelle is specific for one analyte.

In some aspects of the disclosed methods, the sample includes no more than 50, no more than 25, no more than 10, or no more than 5 particles of the analyte. In one non-limiting example, the sample includes as little as one particle of the analyte (for example, one SARS-CoV-2 virion).

Also provided are computer-readable storage media that store computer-executable instructions, which when executed by the processor, cause a computer to perform at least some of the steps of the disclosed methods. For example, the methods can include receiving data from an electrometer coupled to a sensor comprising the imprinted micelle, and comparing the data to background data to estimate particle-micelle interactions, for example using a processor. Also provided are apparatuses that include memory and the processor configured to execute the computer-executable instructions stored on computer-readable storage media.

The foregoing and other features of this disclosure will become more apparent from the following detailed description of several aspects which proceeds with reference to the accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1E: (FIG. 1A) Fluoromicrograph of imprinted micelles floating in suspension at the time of analyte (methanol) introduction to the liquid (out of frame). (FIG. 1B) Fluoromicrograph of imprinted micelles as the analyte diffuses into the imprinted micelle suspension. The released ion exchange appears as concentric rings of luminescence. (FIG. 1C) The resultant liquid suspension of imprinted micelles after mixing with the analyte. Few imprinted micelles are seen. (FIG. 1D) The electronic signal as a function of serial dilutions of analyte particles. DIW, deionized water. (FIG. 1E) The electronic signal after 2 sigma background elimination of very dilute (>pg/ml) analyte samples.

FIGS. 2A-2D: (FIG. 2A) The electronic signal of an imprinted micelle-analyte interaction versus time and compared to various backgrounds. (FIG. 2B) The magnitude of the electronic signal of the imprinted micelle-analyte interaction versus a large number of controls. (FIG. 2C) The fast-Fourier-transform of the imprinted micelle-analyte interaction versus a control to demonstrate the stochastic nature of the interactions. (FIG. 2D) The relative specificity of the imprinted micelle-analyte interaction to similar particles in both event magnitude and total response. The inlays are transmission electron microscopy (TEM) images of each type of particle. In FIGS. 2A and 2C, DIW is deionized water.

FIGS. 3A-3D: (FIG. 3A) Image of the sampling chamber where ambient air is mixed with generated aqueous aerosols. The relative flow of analyte particles is overlayed on the image. (FIG. 3B) Image of the condensation chamber where the analyte-filled aerosol is captured in liquid water while carrier gas and air are removed from the system. An arrow shows that samples are taken from the liquid and injected into the electrochemical detection cell. (FIG. 3C) The near-time electrochemical results showing a preliminary mixing phase followed by strong signal as the analyte liquid is introduced into the cell. (FIG. 3D) The longer-time life of the analyte-micelle signal.

FIG. 4: Measurement of the signal between the control deionized water (DIW) and the micelle-analyte interaction.

FIGS. 5A-5D: (FIG. 5A) Ultraviolet (UV)-inactivated SARS-CoV-2 micelle response to UV-inactivated SARS-CoV-2. (FIG. 5B) Response to pseudotyped particles of SARS-CoV-2. (FIG. 5C) Pseudotyped particles of SARS-CoV-2 micelle response to pseudotyped particles of SARS-CoV-2. (FIG. 5D) Response to UV-inactivated SARS-CoV-2.

FIG. 6: Example cyclic voltammetry of the micelles in water at the same concentration as was used for analyte analysis. Ten mV/s sample speed with (1) being the initial look and (2) being return.

FIG. 7: Schematic depicting an imprinted micelle (left) with multiple molecularly imprinted particles specific for a virus (such as SARS-CoV-2) on the surface of the micelle. Upon binding of the virus, an electrolyte solution within the imprinted micelle is released (right, labelled as “Electrolyte Release”), generating an electronic signal that can be measured using commercially available electronics.

FIG. 8: Schematic for visualization of the synthesis steps and reaction for the functionally imprinted micelles.

### DETAILED DESCRIPTION

### I. Abbreviations

AuNP gold nanoparticle
COVID-19 coronavirus disease 2019
DIW deionized water
IMS inner micelle structure
MFM macromolecularly functional monomers
PEG polyethylene glycol
PTFE polytetrafluoroethylene
RT-PCR reverse transcription-polymerase chain reaction
SARS-CoV-2 severe acute respiratory syndrome coronavirus 2
SM sensitized micelle
TEM transmission electron microscopy
UV ultraviolet

### II. Terms and Methods

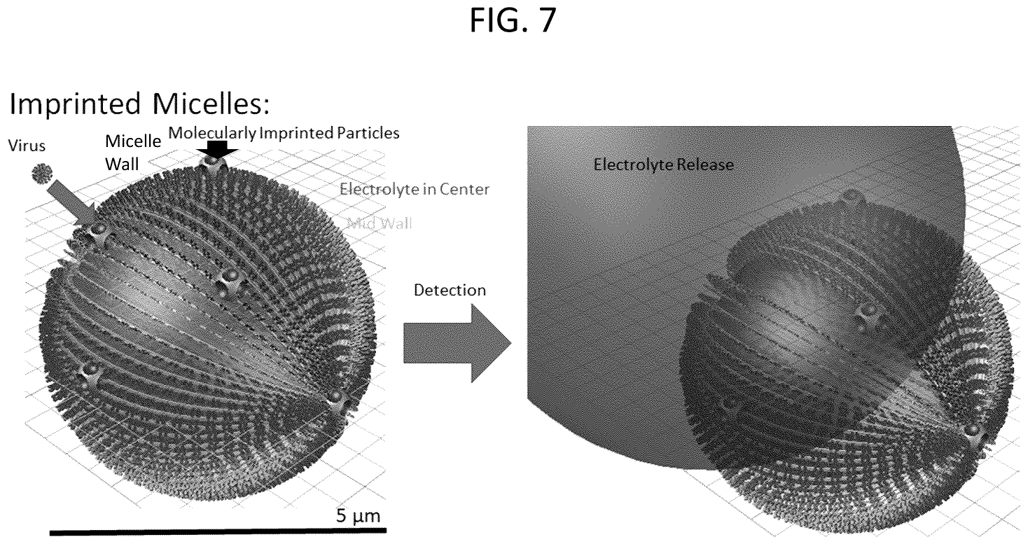

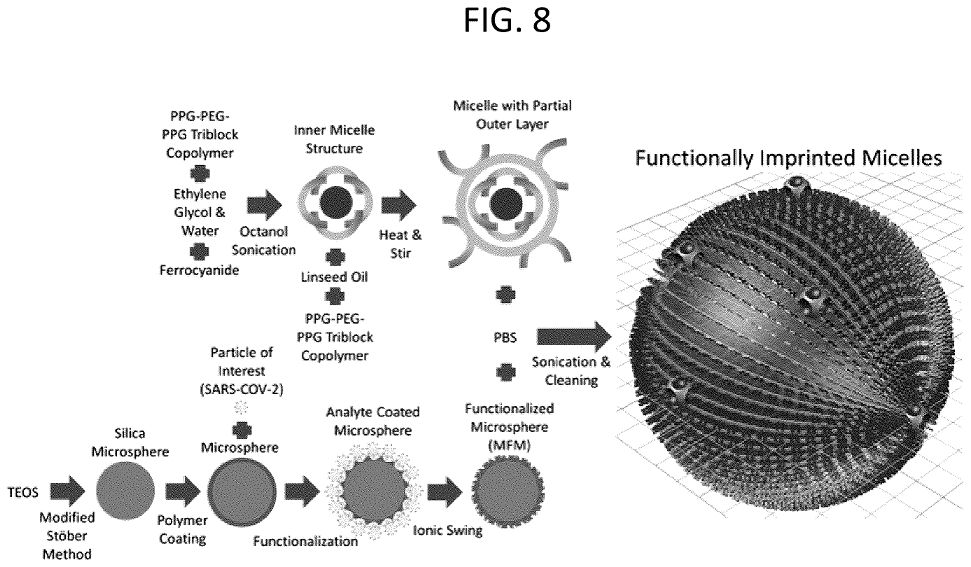

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “a pathogen” includes singular or plural antigens and can be considered equivalent to the phrase “at least one pathogen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various aspects, the following explanations of terms are provided:
Aerosol: A suspension of fine solid particles or liquid droplets in a gas (such as air).
Analyte: A target molecule to be detected, quantified and/or analyzed. Exemplary analytes include, but are not limited to, pathogens (e.g., viruses, virus particles, bacteria, fungi, spores, and portions thereof), proteins (such as one from a virus or bacteria, or a toxin), antigens, antibodies, nucleic acid molecules, chemicals, and explosive particles.
Condensation: A process in which a gas is converted into a liquid.
Contacting: Placement in direct physical association; includes both in solid and liquid form.
Control: A reference standard, for example a positive control or negative control. A positive control is known to provide a positive test result (e.g., a sample known to include an analyte of interest). A negative control is known to provide a negative test result (e.g., a sample known not to include an analyte of interest). However, the reference standard can be a theoretical or computed result, for example a result obtained in a population.
Coronavirus: A large family of positive-sense, single-stranded RNA viruses that can infect humans and non-human animals. Coronaviruses get their name from the crown-like spikes on their surface. The viral envelope is comprised of a lipid bilayer containing the viral membrane (M), envelope (E) and spike (S) proteins. Most coronaviruses cause mild to moderate upper respiratory tract illness, such as the common cold. However, three coronaviruses have emerged that can cause more serious illness and death: severe acute respiratory syndrome coronavirus (SARS-CoV), SARS-CoV-2 (including variants and lineages thereof, such as: alpha (B.1.1.7 and Q lineages); beta (B.1.351 and descendent lineages); delta (B.1.617.2 and AY lineages); gamma (P.1 and descendent lineages); epsilon (B.1.427 and B.1.429); eta (B.1.525); iota (B.1.526); kappa (B.1.617.1); 1.617.3; mu (B.1.621, B.1.621.1) zeta (P.2) and omicron (including BA.1, BA.1.1, BA.2, BA.4, BA.5, BQ1, XBB)), and Middle East respiratory syndrome coronavirus (MERS-CoV). Other coronaviruses that infect humans include human coronavirus HKU1 (HKU1-CoV), human coronavirus OC43 (OC43-CoV), human coronavirus 229E (229E-CoV), human coronavirus NL63 (NL63-CoV).
COVID-19: The disease caused by the coronavirus SARS-CoV-2.
Detect: To determine if a particular agent or analyte is present or absent, and in some examples further includes quantification of the agent/analyte if detected. In some examples, the agent detected is a coronavirus, such as SARS-CoV-2.
Electrochemical cell: A vessel capable of generating electrical energy. In some aspects herein, the electrochemical cell is a glass vessel with a polystyrene shell containing deionized water, and upon release of an electrolyte solution from an imprinted micelle within the vessel, an electrical signal is detected.
Electrolyte solution: A liquid containing ions through which electricity can pass.
High-κ dielectric material: Any material with a high dielectric constant (kappa, κ) relative to silicon dioxide. High-κ dielectric materials include, but are not limited to, hafnium silicate (HfSiO₄), zirconium silicate (ZrSiO₄), hafnium dioxide (HfO₂), zirconium dioxide (ZrO₂), aluminum oxide (Al₂O₃), silicon nitride (Si₃N₄), titanium dioxide (TiO₂), scandium(III) oxide (Sc₂O₃), yttrium oxide (Y₂O₃), lanthanum oxide (La₂O₃), lutetium((III) oxide (Lu₂O₃), niobium(V) oxide (Nb₂O₅), tantalum pentoxide (Ta₂O₅) and HfZrO₄ (see, e.g., Clark, Materials (Basel) 7(4):2913-2944, 2014).
Imprinted micelle: A micelle having one or more analyte imprints (e.g., imprints that correspond to the one or more analytes), such as one or more molecularly imprinted particles, on its surface (e.g., see FIGS. 7 and 8). In some examples herein, the micelle has about 5 to about 10 analyte imprints, such as about 5 to about 10 molecularly imprinted particles, on its surface.
Micelle: An assembly of phospholipid molecules that have a generally spherical shape in an aqueous solution.
Molecularly imprinted particle (MIP): Particles in which analytes of interest have been “imprinted” on the surface. Molecularly imprinted particles contain recognition cavities that are complementary to the shape and size of the analyte of interest, and thereby function as receptors for the analyte. Exemplary particles include those composed of one or more polymers, one or more quantum dots, glass, gold, one or more magnetic materials, or combinations thereof. Exemplary polymers include those containing benzene ring backbones copolymerized with acrylamides. In one example the polymer is polyethylene glycol. Exemplary materials that can be used alone or in combination to generate molecularly imprinted particles include but are not limited to silica nanoparticles, magnetic nanoparticles, gold nanoparticles, polymer nanoparticles (such as one that includes polyethylene glycol), polymer-silica composite nanoparticles, quantum dots, and nanoclusters. In some examples, the outer layer of an MIP is multi-ionic. In one example, an MIP is composed of a polymer particle having attached thereto one or more quantum dots (nanoparticles), such as a polyethylene glycol particle having attached thereto one or more quantum dots (e.g., in a specific orientation). In another example, an MIP is composed of glass microspheres (such as 500-1000 nm in diameter) coated in one or more rigid polymers (such as polymers containing benzene ring backbones copolymerized with acrylamides). In some examples, the particle is at least 10 nm in diameter, such as at least about 20 nm in diameter, such as at least about 50 nm in diameter, such as at least about 100 nm in diameter, such as at least about 200 nm in diameter, such as at least about 500 nm in diameter, or such as at least about 1000 nm in diameter, for example 10-5000 nm, 10-2500 nm, 10-1000 nm, 10-750 nm, 10-500 nm, 10-250 nm, 10-100 nm, 10-50 nm, 100-1000 nm, 100-5000 nm, 100-500 nm, 100-200 nm, or 50-500 nm in diameter. In some examples, the MIP includes a silica microsphere about 100 nm in diameter, which may be coated with a polymer outer layer. Methods for producing molecularly imprinted polymers have been previously described (e.g., see Niu et al., Microchim Acta 183:2677-2695, 2016; Qian et al., Analytica Chimica Acta 884:97-105, 2015; Saylan et al., Sensors 17:898, 2017; Chen et al., Chem Soc Rev 45:2137, 2016; Ahmad et al., Chem Mater 27:5464-5478, 2015; U.S. Patent Application Publication No. 2022/0167873).
Pathogen: A biological organism or agent that causes disease or illness to its host. Pathogens include, for example, bacteria, viruses, fungi, helminths, protozoa and other parasites. Pathogens can also be referred to as infectious agents.
Plurality: Any number that is more than one. In some aspects herein, a “plurality of analytes” means at least 2, at least 3, at least 4, at least 5, at least 6, at least 7, at least 8, at least 9 or at least 10 analytes.
SARS-CoV-2: A coronavirus of the genus betacoronavirus that first emerged in humans in 2019. This virus is also known as Wuhan coronavirus, 2019-nCoV, or 2019 novel coronavirus. Symptoms of SARS-CoV-2 infection include fever, chills, dry cough, shortness of breath, fatigue, muscle/body aches, headache, new loss of taste or smell, sore throat, nausea or vomiting, and diarrhea. Patients with severe disease can develop pneumonia, multi-organ failure, and death. The time from exposure to onset of symptoms is approximately 2 to 14 days. The SARS-CoV-2 virion includes a viral envelope with large spike glycoproteins. The SARS-CoV-2 genome, like most coronaviruses, has a common genome organization with the replicase gene included in the 5′-two thirds of the genome, and structural genes included in the 3′-third of the genome. The SARS-CoV-2 genome encodes the canonical set of structural protein genes in the order 5′-spike (S)-envelope (E)-membrane (M) and nucleocapsid (N)-3′. The term “SARS-CoV-2” includes variants and lineages thereof, such as, but not limited to, alpha (B.1.1.7 and Q lineages); beta (B.1.351 and descendent lineages); delta (B.1.617.2 and AY lineages); gamma (P.1 and descendent lineages); epsilon (B.1.427 and B.1.429); eta (B.1.525); iota (B.1.526); kappa (B.1.617.1); 1.617.3; mu (B.1.621, B.1.621.1); zeta (P.2); and omicron (including BA.1, BA.1.1, BA.2, BA.4, BA.5, BQ1 and XBB)).
Subject: Living multi-cellular vertebrate organisms, a category that includes both human and non-human animals (such as birds, pigs, mice, rats, rabbits, sheep, horses, cows, and non-human primates).
Solid support: In the context of the present disclosure, a “solid support” refers to a solid structure to which an imprinted micelle can be adhered. In some examples herein, the solid support is a filter, such as a filter with conductive fibers. In some examples herein, the solid support is glass. In some examples herein, the solid support is plastic. In some examples herein, the solid support is metal. In some examples herein, the solid support is composed of multiple types of materials (e.g., glass, metal, electrodes and additively manufactured materials).

### III. Introduction

A need exists for detection of airborne pathogens to control the COVID-19 pandemic and avoid future pandemics, for example in real-time. Described herein is a system developed for the detection of ambient liquid samples containing an analyte of interest, such as severe acute respiratory syndrome coronavirus 2 (SARS-CoV-2), coupled with airborne sampling. In some examples, the methods are performed in real time. The ability to detect SARS-CoV-2 or other pathogens, for example in real-time, allows rapid screening and early detection of virus spread. Existing SARS-CoV-2 detection methods include rapid antigen tests, serological surveys, and reverse transcription-polymerase chain reaction (RT-PCR). However, these methods are not currently compatible with airborne sampling (Kumar et al., Chem. Eng. J. 430(3):132966, 2022). Based on the examples herein using SARS-CoV-2 as a model system, a skilled person will appreciate that the methods can be used to detect other analytes of interest.

One technology that is compatible with airborne sampling is molecular imprinting, which detects compounds of interest, including molecules, proteins, inorganic particles, viruses, and bacteria (Ahmad et al., Chem. Mater. 27(16):5464-5478, 2015; Chen et al., Chem. Soc. Rev. 45(8):2137-2211, 2016; Niu et al., Microchim. Acta 183:2677-2695, 2016; Qian et al., Anal. Chimica Acta 884:97-105, 2015; Saylan et al., Sensors 17(4):898, 2017). Many molecular imprinting detection methods are optically based (Qian et al., Anal. Chimica Acta 884:97-105, 2015) with electronic detection methods being a less-studied aspect of the technology (Saylan et al., Sensors 17(4):898, 2017). To advance imprinting technology to be field-deployable, the present disclosure describes functionalization of imprinting technology to produce a signal that can be detected by commercial electronic systems.

Micelles (O'Reilly et al., Chem. Soc. Rev. 35:1068-1083, 2006) in an electrochemical cell produce measurable electronic signals when they change morphology (Ferreira et al., J. Phys. Chem. C 114(17):7710-7716, 2010; Macka et al., J. Chromatogr. A 1-2:193-199, 2004). The electronic signals from the micelles are read as changes in the properties of the bulk liquid. By incorporating imprinting technology into a micelle, the imprinting technology is imbued with an electronic signal that can be read in near real-time.

The present disclosure specifically describes the integration of imprinting technology and micellular components to provide a system that allows for detection of airborne analytes of interest by commercially available electronics. Specifically, it is demonstrated that (1) the release of picomolar amounts of analyte in a sub-mL electrochemical cell is detected by commercially available fA-scale electronics; (2) the incorporation of 100 nm scale imprinted particles at an average loading of 5-10 molecularly imprinted particles per micelle into the outer wall of a dual-layer micelle (with an average diameter of 5 μm) destabilizes the micelle when brought into contact with analytes that correspond to the imprint on the particles; and (3) imprinted micelle specificity is applicable to a biological system, as exemplified by the specific detection of SARS-CoV-2. In addition, the examples provided herein show that the interaction between a target analyte (e.g., viral particles) and molecularly imprinted particles specific for the target analyte and which are embedded into a micelle releases indicators (salts/electrolytes) from the imprinted micelle and generates a signal within an electrochemical cell (see FIG. 7). The present disclosure is the first demonstration of airborne particulate detection with micelles containing an imprint specific for the analyte of interest.

### IV. Methods for Detection of Airborne Analytes

Provided herein are methods for detecting an airborne analyte. In some aspects, the methods include contacting an ambient air sample suspected of containing the analyte with an aqueous aerosol and subjecting the air sample and aqueous aerosol to condensation, thereby producing a liquid-analyte solution; contacting the liquid-analyte solution with an imprinted micelle, wherein the imprinted micelle includes at least one imprint of the analyte and contains an electrolyte solution; and detecting an electrochemical signal produced upon binding of the analyte to the imprinted micelle, releasing the electrolyte solution, thereby detecting the airborne analyte. The disclosed methods can be adapted for the detection of any airborne analyte by generating a micelle containing at least one imprint of the analyte of interest. A skilled person will appreciate that multiple analytes can be detected, for example by using multiple imprinted micelles, each containing at least one imprint of each analyte to be detected. In some examples, a single imprinted micelle includes imprints of at least two different analytes. In some examples, a single imprinted micelle includes 1 to 15, such as 3 to 12, or 5 to 10 imprints of the analyte. In specific examples, a single imprinted micelle includes 5, 6, 7, 8, 9 or 10 imprints of the analyte.

In some aspects, the airborne analyte is a pathogen, such as a virus, a bacterium or a fungus. In some examples, the imprint is of the pathogen itself, or a portion thereof, such as a protein, such as a spike viral protein.

For aspects in which the analyte is a virus, the virus can be an RNA virus (such as a positive-sense RNA virus, a negative-sense RNA virus, or a double-stranded RNA virus), or a DNA virus (such as a single-stranded DNA virus or a double-stranded DNA virus).

Examples of viruses that can be detected with the disclosed methods include, but are not limited to viruses in the following virus families: Retroviridae (for example, human immunodeficiency virus (HIV), human T-cell leukemia viruses); Picornaviridae (for example, poliovirus, hepatitis A virus, enteroviruses, human coxsackie viruses, rhinoviruses, echoviruses, foot-and-mouth disease virus); Caliciviridae (such as strains that cause gastroenteritis, including Norwalk virus); Togaviridae (for example, alphaviruses (including chikungunya virus, equine encephalitis viruses, Semliki Forest virus, Sindbis virus, Ross River virus, rubella viruses); Flaviridae (for example, hepatitis C virus, dengue viruses, yellow fever viruses, West Nile virus, St. Louis encephalitis virus, Japanese encephalitis virus, Powassan virus and other encephalitis viruses); Coronaviridae (for example, coronaviruses, severe acute respiratory syndrome (SARS) virus, SARS-CoV-2, Middle East respiratory syndrome (MERS) virus; Rhabdoviridae (for example, vesicular stomatitis viruses, rabies viruses); Filoviridae (for example, Ebola virus, Marburg virus); Paramyxoviridae (for example, parainfluenza viruses, mumps virus, measles virus, respiratory syncytial virus); Orthomyxoviridae (for example, influenza viruses); Bunyaviridae (for example, Hantaan viruses, Sin Nombre virus, Rift Valley fever virus, bunya viruses, phleboviruses and Nairo viruses); Arenaviridae (such as Lassa fever virus and other hemorrhagic fever viruses, Machupo virus, Junin virus); Reoviridae (e.g., reoviruses, orbiviurses, rotaviruses); Birnaviridae; Hepadnaviridae (hepatitis B virus); Parvoviridae (parvoviruses); Papovaviridae (papilloma viruses, polyoma viruses, BK-virus); Adenoviridae (adenoviruses); Herpesviridae (herpes simplex virus (HSV)-1 and HSV-2; cytomegalovirus; Epstein-Barr virus; varicella zoster virus; Kaposi's sarcoma herpesvirus (KSHV); and other herpes viruses, including HSV-6); Poxviridae (variola viruses, vaccinia viruses, pox viruses such as monkeypox); and Iridoviridae (such as African swine fever virus); Astroviridae; and unclassified viruses (for example, the etiological agents of spongiform encephalopathies, the agent of delta hepatitis (thought to be a defective satellite of hepatitis B virus).

In some examples, the virus detected with the disclosed methods is a respiratory virus, such as a coronavirus, an influenza virus, a parainfluenza virus, a respiratory syncytial virus, an adenovirus, a rhinovirus, or a metapneumovirus.

In specific examples, the respiratory virus detected with the disclosed methods is a human coronavirus, such as severe acute respiratory syndrome coronavirus 2 (SARS-CoV-2), SARS-CoV, Middle East respiratory syndrome coronavirus (MERS-CoV), human coronavirus HKU1 (HKU1-CoV), human coronavirus OC43 (OC43-CoV), human coronavirus 229E (229E-CoV), or human coronavirus NL63 (NL63-CoV). As used herein, “SARS-CoV-2” encompasses the original Wuhan strain of SARS-CoV-2 and all variants of SARS-CoV-2, such as an alpha (B.1.1.7 and Q lineages); beta (B.1.351 and descendent lineages); delta (B.1.617.2 and AY lineages); gamma (P.1 and descendent lineages); epsilon (B.1.427 and B.1.429); eta (B.1.525); iota (B.1.526); kappa (B.1.617.1); 1.617.3; mu (B.1.621, B.1.621.1), zeta (P.2), or omicron (including BA.1, BA.1.1, BA.2, BA.4, BA.5, BQ1, XBB) variant.

In other specific examples, the respiratory virus detected with the disclosed methods is an influenza virus, such as an influenza A virus (such as H1N1 (e.g., 1918 H1N1), H1N2, H1N7, H2N2 (e.g., 1957 H2N2), H2N1, H3N1, H3N2, H3N8, H4N8, H5N1, H5N2, H5N8, H5N9, H6N1, H6N2, H6N5, H7N1, H7N2, H7N3, H7N4, H7N7, H7N9, H8N4, H9N2, H10N1, H10N7, H10N8, H11N1, H11N6, H12N5, H13N6, or H14N5), an influenza B virus or an influenza C virus.

In some aspects in which the analyte is a bacterial pathogen, the bacterial pathogen includes, but is not limited to, Helicobacter pylori, Escherichia coli, Vibrio cholerae, Borelia burgdorferi, Legionella pneumophilia, Mycobacteria sps (such as. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, corynebacterium sp., Erysipelothrix rhusiopathiae, Clostridium perfringers, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Bordetella pertussis, Shigella flexnerii, Shigella dysenteriae and Actinomyces israelli.

In some aspects in which the pathogen is a fungal pathogen, the fungal pathogen detected with the disclosed methods includes, for example Aspergillus sp., Cryptococcus neoformans, Histoplasma capsulatum, Coccidioides immitis, Blastomyces dermatitidis, Chlamydia trachomatis or Candida sp. (e.g., C. albicans).

In other aspects, the airborne analyte detected with the disclosed methods is a toxin, such as anthrax toxin, abrin, ricin, Pseudomonas exotoxin, diphtheria toxin, or botulinum toxin.

In other aspects, the airborne analyte detected with the disclosed methods is a protein. For example, the protein can be a protein from a virus, bacterium or fungus, such as any one of the viruses, bacteria and fungi listed herein.

In other aspects, the airborne analyte is an explosive particle, such as nitroglycerin, black power, gun powder, dynamite, pentaerythritol tetranitrate, or ammonium nitrate. The explosive particle can also include a nitro-organic (e.g., DMDNB, DNT, NM, PETN, PLX, RDX, RDX, HMX, TATB, TNT, ammonium nitrate, nitroguanidine, mercury fulminate, urea nitrate, picric acid, trinitroanline, ETN, DAAF), a peroxide (e.g., H₂O₂, HMTD, TATP, MEKP), cyclohexanone, or a fuel/oxidizer mixture (e.g., AN, AN-FO, AN-NM, KClO-S, KClO₃-FO).

In other aspects, the airborne analyte detected with the disclosed methods is an environmental pollutant, an organic molecule, an inorganic particle, a chemical, or a pharmaceutical (e.g., fentanyl, cocaine, a methamphetamine, an opiate or heroin).

In some aspects of the disclosed methods, the imprinted micelle is a double-layered micelle, and the outer layer of the micelle includes at least one molecularly imprinted particle specific for the analyte. In some examples, molecularly imprinted particles are composed of one or more polymers, one or more quantum dots, glass, gold, magnetic materials, or combinations thereof. In some examples, molecularly imprinted particles have a core composed of at least one substance, such as gold or glass, and an outer layer attached or bound to the core (such as a coating composed of one or more polymers, such as one or more rigid polymers). In some examples, the at least one molecularly imprinted particle includes silica nanoparticles, magnetic nanoparticles, gold nanoparticles, polymer nanoparticles, polymer-silica composite nanoparticles, quantum dots, or nanoclusters. In some examples, the at least one molecularly imprinted polymer includes 1 to 15, such as 3 to 12, or 5 to 10 molecularly imprinted particles per micelle. In specific examples, the imprinted micelle includes 5, 6, 7, 8, 9 or 10 molecularly imprinted particles specific for the analyte.

In some aspects of the disclosed methods, the liquid-analyte solution and imprinted micelle are contacted in an electrochemical cell. In some examples, the electrochemical cell is made up of a glass vessel containing deionized water. In particular examples, the glass vessel is coated with polystyrene, polytetrafluoroethylene (PTFE), alumina, silica or a high-κ dielectric material.

In some examples, the imprinted micelle is adhered to a solid support in the electrochemical cell. In some instances, the solid support is coupled to a conductive fiber filter.

In some aspects of the disclosed methods, the electrolyte solution is any ionic solution that is 10-fold more concentrated than the liquid in the outer aqueous solution. In some examples, the electrolyte solution includes ferrous cyanide, sodium chloride, or fluorescein.

In some aspects of the disclosed methods, the method is a multiplex method, and the imprinted micelle includes imprinted micelles specific for a plurality of analytes. In some examples, each imprinted micelle has at least one molecularly imprinted particle (such as one that includes one or more polymers, metals, glass and/or nanoparticles) specific for each analyte. In other examples, each imprinted micelle is specific for one analyte.

In some examples of the multiplex method, the plurality of analytes includes at least two, at least three, at least four, at least four or at least five different analytes (such as viruses).

In some aspects of the disclosed method, the sample includes no more than 50, no more than 25, no more than 10, or no more than 5 particles of the analyte. In specific examples, the sample includes at least 5, at least 10, at least 20, at least 50, at least 75 or at least 100 molecules of the analyte.

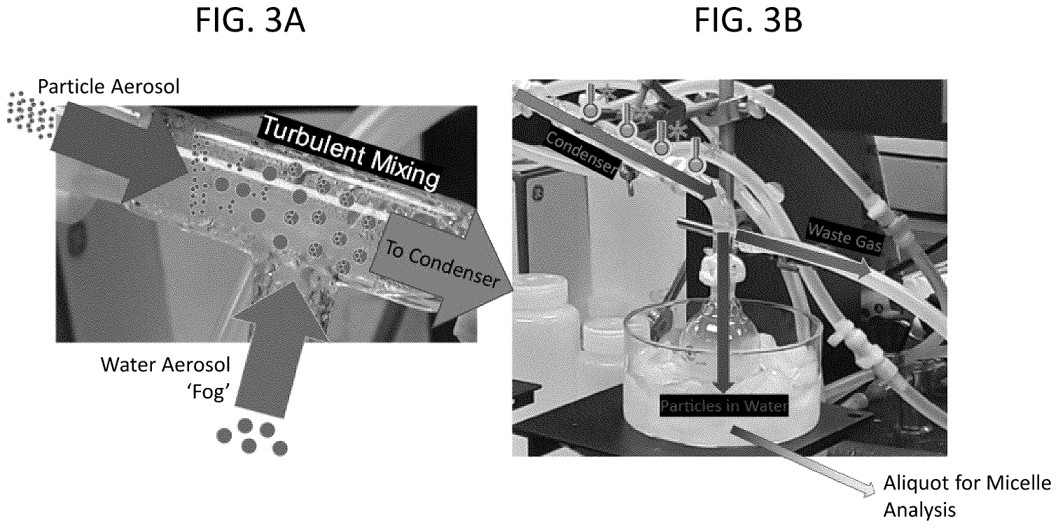

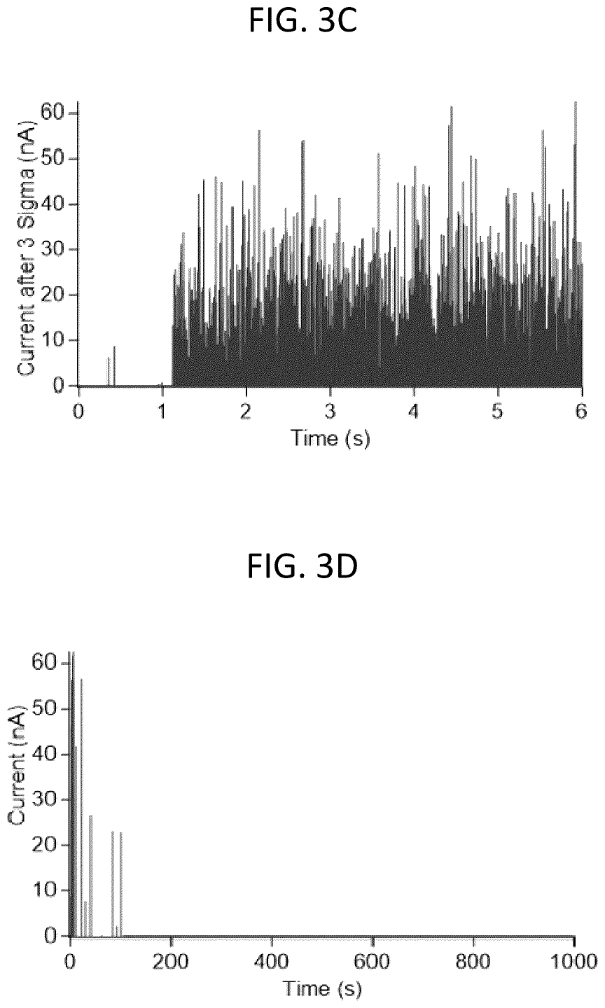

In alternative aspects, the detection method is modified to use a nebulizer-like system. Such a system can be utilized as a replacement to the turbulent mixing and condensing setup shown in FIG. 3A. In some examples, the nebulizer has a high flow rate of air, about 1-1000 liters per minute, with a solution rate of about 0.1-10 mL per minute. The high flow rate of air while the aqueous solution is being sputtered enables mixing and carries the particulate, such as SARS-CoV-2, into a collection vessel as displayed in FIG. 3B for periodic aliquot sampling or into a flow through system in which the collection vessel is constantly being monitored for the analyte (such as SARS-CoV-2).

### V. Sensor Electronics

In some examples, an analyte sensor comprising at least one disclosed micelle is coupled with data acquisition electronics (e.g., an electrometer) to acquire signal data from the analyte sensor. Analysis of the analytes can proceed by comparing the acquired signal data to various backgrounds. In some examples, the data acquisition hardware is coupled to an analyte sensor to apply varying voltages to generate plural current measurements over time. The time-domain current data can be converted to the frequency domain (e.g., using a Fast Fourier Transform (FFT)) and the resulting magnitude and total response can be compared to control data.

The following examples are provided to illustrate particular features of certain aspects of the disclosure, but the scope of the claims should not be limited to those features exemplified.

### EXAMPLES

The studies described in the Examples were performed to demonstrate that the integration of imprinted technology and micellular components can provide a system that allows for detection of analytes (e.g., particles or compounds of interest) by commercially available electronics. It was proposed that: (1) the release of picomolar amounts of analyte in a sub-mL electrochemical cell can be detected by commercially available fA-scale electronics; (2) the incorporation of 100 nm scale imprinted particles at an average loading of 5-10 particles per micelle into the outer wall of a dual-layer micelle (average diameter of 5 μm) can destabilize the micelle when brought into contact with analytes that have been imprinted on the particles; and (3) imprinted particle specificity can be applied into a biological system, such as for the detection of a virus (for example, SARS-CoV-2).

### Example 1: Materials and Methods

### Reagents

Ethanol (CAS: 64-17-5, 99.5%+), tetraethyl orthosilicate (TEOS, CAS: 78-10-4, 99%), ammonium chloride (CAS: 12125-02-9, 99.5%), toluene (CAS: 108-88-3, 99.5%), g-methacryloxypropyltrime-thoxysilane (MPS, CAS: 2530-85-0, 97%+), N-Isopropylacrylamide (NiPAm, CAS: 2210-25-5, 99%+), 1-(chloromethyl)-4-ethenyl-benzene (CMS, CAS: 7398-44-9, 99%), dimethyl sulfoxide (DMSO, CAS: 67-68-5, 99%), isopropanol (CAS: 67-63-0, 99.5%+), azobisisobutyronitrile (AlBN, CAS: 78-67-1, 98%), diethyl ether (CAS: 60-29-7, 99.7%), 1-vinylimidazole (VIM, CAS: 1072-63-5, 99%+), phosphate buffer solution (PBS, 1.0 M pH 7.4, P3619), ammonium persulfate (APS, CAS: 7727-54-0, 98%+), N,N,N,N-tetramethylenebis(acrylamide) (TEMED, CAS: 2956-58-3, >90%), sodium chloride (NaCl, CAS: 7647-14-5, 99%+), ethylene glycol (EG, CAS: 107-21-1, 99%+), poly(ethylene glycol)-block-poly(propylene glycol)-block-poly(ethylene glycol) (PEG-PPG-PEG, CAS: 9003-11-6, 95%+), potassium ferrocyanide trihydrate (CAS: 14459-95-1, 98.5%+), octanol (CAS: 111-87-5, 99%+), linseed oil (CAS: 68553-15-1, QL 200), polyethylene glycol (PEG, CAS: 25322-68-3, 200 kDa, QL 200), gold chloride (CAS: 16961-25-4, 99.9%+), zinc chloride (CAS: 7646-85-7, 99.995%), sodium borohydride (CAS: 16940-66-2, 98%+), and sodium hydroxide (NaOH, CAS: 1310-73-2, 98%+) were purchased from Millipore Sigma or were acquired from Pacific Northwest National Laboratory (PNNL) dry storage and used without further purification.

### Biological and Inorganic Particulate Reagents

UV light-inactivated severe acute respiratory syndrome coronavirus 2 (SARS-CoV-2) viral stocks were prepared as follows. Under biosafety level three containment and using appropriate personal protective equipment, confluent monolayers of Vero E6 cells were infected with high titer SARS-CoV-2 stock (BEI Resources NR-52284) for 35 hours at 37° C. with 5% carbon dioxide in Minimal Essential Media (Gibco 11095080) containing 4% fetal bovine serum (Cytiva Hyclone SH3007003HI) and 1× antibiotic/antimycotic (Gibco 15240112). As part of the natural infection cycle, infectious SARS-CoV-2 is released into the media on top of infected cells. Supernatant (media) from infected cells was collected and gently centrifuged (Sorvall Legend Biocontainment rotor at 500×g for 5 minutes) to remove cellular debris. Supernatant (in 1 mL aliquots) was added to each well in a six-well plate (Corning 3506) and exposed to UV treatment for 10 minutes. The plate was rotated 1800 and the UV treatment was repeated to inactivate SARS-CoV-2 virions. Following treatment, the inactivated virus was collected into 2 mL free standing o-ringed tubes and frozen at −80° C. The UV treatment was repeated until all the media from the infected flask had been inactivated. The UV lamp (UVP 95-0007-06 Model UVGL-58 6-watt UV lamp) used was brand new and was ˜11.43 cm from the six-well plate. The estimated dose was ˜1462 mJ/cm² at 254 nm. UV-inactivated media was evaluated by plaque assay to confirm all replication-competent SARS-CoV-2 virions were rendered no longer infectious. Vero E6 cells were plated in six-well plates and serial dilutions of UV-treated and non-treated SARS-CoV-2 samples in 1× sterile Dulbecco's phosphate buffered saline (Gibco 14190250) were added to the appropriate wells and covered with Dulbecco's minimal essential media (Gibco 11965092), 4% fetal bovine serum, 1× antibiotic/antimycotic, and 0.8% low melting point agarose (Lonza SeaKem LE Agarose 50002). Plaque assay plates were assessed 72 hours post-infection with 1× neutral red stain (Invitrogen N3246). Following Institutional Biosafety Committee approval of the UV inactivation protocol, samples were removed to biosafety level two for further studies.

### SARS-CoV-2 Spike Pseudotyped Particles

Bacterial plasmid constructs containing fragments of the lentiviral vector and SARS-CoV-2 spike gene were transformed into chemically competent E. coli (TOP10 Invitrogen C404003) using heat shock; plasmid confirmation was performed by restriction enzyme digestion screening; selected clones were grown in liquid media cultures under antibiotic selection; and plasmid extraction was performed using a Qiagen miniprep kit (Qiagen 27106) according to the manufacturer's instructions. Under biosafety level two conditions, human embryonic kidney 293T cells expressing human angiotensin converting enzyme 2 (ACE2; BEI Resources NR-52511), the SARS-CoV-2 receptor, were expanded and plated in Dulbecco's minimal essential media (Gibco 11965092) supplemented with 10% fetal bovine serum (Cytiva Hyclone SH3007003HI), 2 mM L-glutamine (Gibco 25030081) and 1× penicillin streptomycin (Gibco 10378016; D10 growth media) at 5×10⁵ cells per well for a six-well plate. Between 16 and 24 hours post-plating, 7.5 mL LIPOFECTAMINE™ 3000 (Invitrogen L3000015) in 125 mL Opti-MEM I media (Gibco 31985070) was mixed with 1 mg plasmid DNA (lentivirus backbone/luciferase/green fluorescent protein, BEI Resources NR-52516), 0.34 mg spike glycoprotein (BEI Resources NR-52514), 0.22 mg helper Rev1b (BEI Resources NR-52519), 0.22 mg helper Tat1b (BEI Resources NR-52518), 0.22 mg helper Gag/Pol (BEI Resources NR-52517) in 125 mL Opti-MEM I media with 5 mL P3000 reagent (provided with the LIPOFECTAMINE™ 3000) per well and used to rescue SARS-CoV-2 pseudotyped particles. The following morning, the DMEM plus transfection reagent and DNA was removed and 2 mL D10 growth media was added. Media were harvested from the transfected cells at 60 hours post-transfection and filtered through 0.45 mM surfactant free cellulose acetate (SFCA) low protein binding filter (Corning 431220) prior to storage at −80° C. Human coronavirus NL63 stock: LLCMK2 cells (non-human primate kidney cells) were grown to 80% confluence and inoculated with 500 μL clinical isolate of human coronavirus NL63 (HCoV-NL63) obtained from BEI Resources (NR-470) and incubated at 32° C. until half of the cells were involved in cytopathic effect. Supernatant was removed from the infected cells, gently pelleted at 500×g for 5 minutes to remove cellular debris, aliquoted and frozen at −80° C. until tested (Donaldson et al., J. Virol. 82(23):11948-11957, 2008). Rabbit anti-guinea pig IgG whole molecule antibodies polyclonal secondary antibody (Abnova Corporation PAB9253) and luminescent particular tracers for fluorescent video imaging were collected from stores of (Hubbard et al., MRS Commun. 10:594-599, 2020) and functionalized with ascorbic acid (Vit.-C, CAS: 50-81-7, 99%) by ligand exchange (Choi et al., Nano. Lett. 21(14):6057-6063, 2021).

### Sensitized Micelle Synthesis

Prior to incorporation into the outer micelle wall, the functionalized silica spheres were prepared similarly to Qian et al. (Anal. Chimica Acta 884:97-105, 2015) as follows, and depicted in FIG. 8.

Silica Sphere Synthesis. 45.5 mL of ethanol and 4.5 mL of TEOS were mixed in ambient environment after which a solution of 16.2 mL of ethanol, 26.3 mL of deionized water (DIW), and 7.5 mL of ammonium chloride were added. The solution was vigorously stirred at 10° C. and incubated for 2 hours. Then, 150 mL of toluene was added and 1 mL of MPS was added under argon and stirred for 48 hours at 50° C. The particles were centrifuged (7000 rpm) and washed separately in ethanol, DIW, and toluene. Lastly, the particles were dried under vacuum at 60° C. for 24 hours and stored under argon.

Macromolecularly Functional Monomers (MFMs). In a 25 mL flask, a stir bar, 2.26 g of NiPAm, and 1.02 g of CMS were added. The powders were dissolved in 8 mL of 50 vol % DMSO and isopropanol. Then 3 mg of AlBN were added to the solution, which was deoxygenated by argon purge for 1 hour at ambient temperature. The solution was then heated to 75° C. for 8 hours. The MFMs were precipitated by addition of 50 mL of freezing diethyl ether. The solid was dissolved in 10 mL of DMSO and 0.63 g of VIM was added dropwise at 50° C. The solution was stirred for 8 hours at 50° C. and then the solids were precipitated with freezing diethyl ether and washed with 100 mL of ether. Lastly, the solid MFMs were dissolved in 5 mL of DIW.

### Particle Imprinting of MFM-Silica and Gold-PEG Particles (IMPs)

### Biology

0.2 mg of the particle to be tested were suspended in 50 mL of PBS to which 20 mg of MFM was added. The suspension was incubated at 30° C. for 3 hours. The liquid was degassed under vacuum for 10 minutes and then purged with argon for 10 minutes. To the liquid, 4 μL of APS and 2 μL of TEMED were added and stirred violently at 30° C. for 24 hours. The particles were collected by centrifugation (7000 rpm, 1 hour) and washed separately with DIW and then 0.5 M NaCl solution. The resultant suspension was stored in 0.5 mL of DIW under argon at 5° C.

### Biology (Virus-Like Pseudo-Particles and Inactivated Virions)

Pseudotyped particles with SARS-CoV-2 spike proteins or UV-inactivated SARS-CoV-2 stocks (˜1 mL) were suspended in 2.5 mL of PBS to which 5 mg SiO₂ and 1 mg of MFM were added. The suspension was incubated at 30° C. for 3 hours with vigorous stirring. To the liquid, 10 μL of 0.2% w/v APS and 10 μL of 0.1% v/v TEMED were added and stirred violently at 30° C. for 12 hours. The particles were collected by centrifugation (1750×g, 20 minutes) and washed separately with DIW, 0.5 M NaCl solution, and a final DIW wash. The resultant suspension was stored in 0.5 mL of DIW in a glass vial until added to the SM solution as described below.

### Inorganic

To make the imprinted Au-PEG particles, 100 mg of PEG 200 Da was added to 20 mL PBS and stirred on high for 10 minutes. Five mg of 300 Da PEG was added followed by 5 mg of the desired analyte particle. Gold chloride (20 mg) was added to the beaker, followed by the careful addition of 10 mg sodium borohydride. The solution, a pale yellow from the gold, was heated to 30° C. and slowly stirred. Small black flecks formed in the liquid after 5 minutes. After 1.5 hours, the solution turned a gray-yellow color. After an additional hour, 50 mg of zinc chloride was added and it was cooled to room temperature. This was centrifuged (7000 rpm) for 30 minutes and the solution decanted off. The remaining solid was put in 5 mL of PBS to make the PEG-AuNP lock solution.

### Inner Micelle Structure (IMS)

10 ml of EG and 10 ml of DIW were added to a beaker to which 180 mg of PEG-PPG-PEG was added. The solution was stirred at 500 rpm for 1 minute, to which 5 mg of potassium ferrocyanide trihydrate was added. Five more mL of DIW were added and the suspension was stirred at 80° C. for 10 minutes at 500 rpm. 7.5 mL of the IMS suspension was added to 20 mL of octanol and sonicated for 30 minutes (40 kHZ, 40 W). Then it was spun in a centrifuge at 7000 rpm for 2 hours until the three layers were observed. The top layer was octanol slightly cloudy with micelles, the middle layer was murky, and the bottom layer was water. The top layer was decanted and collected to finish the first micelle wall solution.

Incorporation of MFM-Silica into Outer Wall of Sensitized Micelles

20 mL of linseed oil and 325 mg of PEG-PPG-PEG were stirred vigorously for 10 minutes at 60° C. (in biological safety cabinet [BSC] at 80° C. for 20 minutes or until dissolved). 20 mL of the IMS suspension was added and stirred vigorously (1500 rpm) for 10 minutes at 60° C. (in BSC 80° C. for 5 minutes or until dissolved). 200 μL of the IMPs suspension (500 μL of biological IMPs) were added to the oil suspension and stirred vigorously for 5 more minutes at the same temperature. The micellular suspension was removed from heat (in the BSC, the heat block temperature was reduced to 40° C.) and stirred at 1500 rpm for 5 more minutes. Lastly, 50 mL of basic DIW (200 mg NaCl, 50 mg NaOH) was added to the suspension and stirred at 40° C. for 5 minutes, then sonicated (BSC in an ice bath) (40 kHz. 40 W) for 60 min. The suspension was centrifuged at 700 rpm for 3 hours (1750×g for a total of 2 hours) stopping each hour to remove oil from the top of the suspension. The suspension of SMs was stored under ambient conditions (4° C. for biological material) in the water mixture until needed for measurement.

### Electrochemical Cell

The electrochemical testing of the SMs was performed in a 100 μL glass vial with an outer polystyrene shell (The Lab Depot Inc., Catalogue Code: 30111G-1232). Two platinum wires (5% Mo, 50 μm dia.) were inserted into the vial and separated by a small piece of PTFE. 100 μL of DIW (cleaned at 2V for 48 hours) was injected into the vial and held at the analysis voltage from 15 minutes to remove any remaining electrolytes in the bath. When the experimental measurements were run, 5 μL of the micelle solution was injected into the cleaned water, as well as 5 μL of the desired analyte solution.

The cell was held in a rubber-covered steel clamp that completely covered the cell. The clamp provided mechanical stability and radio frequency shielding. The metal platform of the cell was grounded to the electrometer and rested on two 3 mm thick sheets of PTFE plastic for electrical isolation. Several layers of alumina-silicate mat were placed under the PTFE, which isolated the cell from common laboratory vibrations and the ventilation vibration inherent in biological-rated hoods.

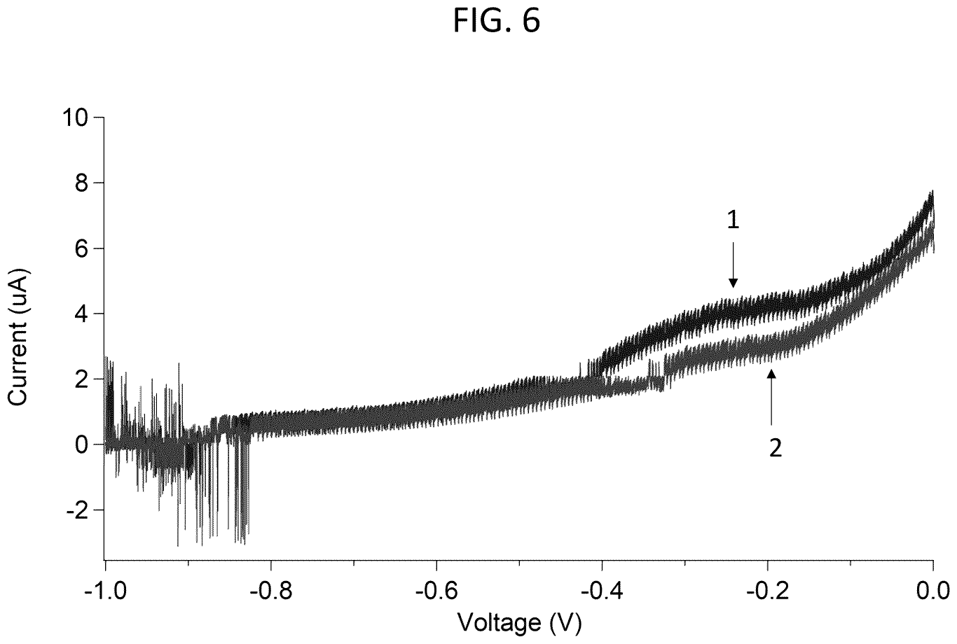

The electrical bias and measurement were performed by a high-resistance electrometer (Keysight Inc., B2985A). Tri-ax cabling (Pamona Inc., Model number: 4725) was used to connect the cell and electrometer. The ground of the electrometer was used for all grounding shield lines so that the cell and electrometer were at the same electrical bias. The electrometer was isolated vibrationally using a rubber mat. The electrometer applied from −0.25 to −0.8 V (typically −0.25 V was used) based on cyclic voltammetry (FIG. 6) performed on the SMs. The electrometer was set from 100 to 100 k samples/s depending on the experiment. Common noise levels achieved in the cell were from 15 to 150 pA, depending on supply-side line noise.

### Photoluminescent Video

Luminescent video was taken on an OMAX EPI-2500× fluoromicroscope under 100 W of broadband UV irradiation. Addition of analytes to a 20 μL drop of micelles on a glass slide was imaged in the microscope and video was taken from 10-0.5 frames/s.

### Airborne Particle Sampling and Condensation

The aerosol condensation setup was constructed of standard laboratory glassware (see FIGS. 3A-3B). One system inlet allowed for the analyte particle input, the other for the generated aqueous aerosol fog (AGPtek, 100 W, 20 kHz) to bind with the air particles. The fog was generated from deionized water >17.8 MOhm. A shared nitrogen gas valve (1-10 L/minSTP) was used to create the air flow throughout the system, carrying both the input analyte particle sample and the particle-collecting fog. Low air flow was used, allowing just enough flow to begin visually generating a vortexing behavior where the two inputs mixed at a three-way adapter glassware. The gas flow then traveled through a sloped condenser glassware, through which a pumped refrigerant at 0° C. flowed through the outer vessel of the condenser. This encouraged the condensation of the fog with the aerosol particle, leading to a two-neck round bottom flask where the sample was collected. This flask is also submerged in an ice bath to help encourage any final condensation of the carrier fog. The second neck of the flask is an outlet to a secondary collection bottle filled with quartz wool to collect remaining fog not condensed. This setup has been measured to collect approximately 0.45 g/minute of condensed fog.

### Data Acquisition and Analysis

Data can be acquired from a sensor coupled to suitable data acquisition electronics. For example, an electrometer can be used to apply a range of voltages to the sensor using cyclic voltammetry. The electrometer generates current samples (e.g., 100 to 100 k samples/s) over a time period (e.g., 1 to 10 s). The current samples are converted to digital values, which can be produced in any suitable format (for example, as a comma separated value (.csv) file). Examples of suitable techniques for measuring current to/from the sensor include well-known techniques using shunt resistors, transformers, or a Hall effect sensors.

In some examples data analysis is undertaken using the following actions performed with a computer:
1. Data is read from .csv files obtained from an electrometer coupled to a sensor introduced to analytes.
2. The largest background (a sample containing just micelles and water in the vial) is determined by the largest standard deviation.
3. The maximum and minimum values are calculated from the background values
4. The maximum and minimum values are subtracted from the analyzed data. In other words, any signal values between the maximum and minimum values of the background values are deleted)
5. The remaining signal is then plotted and used as a time comparison of detection (e.g., in nanoamperes (nA) vs time (s))
6. Integrated signal can be used to estimate the particle-micelle interactions.
7. The data can be output (e.g., to a computer display, printer, or plotter) for user analysis.

In the preceding example, the maximum and minimum values of the background are used as the limits for background subtraction, but in other examples, other suitable limits can be used, for example, twice the standard deviation of the background values (2a).

A Python script used to automate an example of data processing in accordance with the disclosed technology is reproduced below as Listing 1:

```
import pandas as pd
from pathlib import Path
#Grabs all .csv data file from designated folder and sorts by time acquired
folder = Path(r”C:\Users\user\image_folder “)
files = list(folder.glob(“*.csv”))
files.sort(key= lambda x : x.stat( ).st_mtime)
df = pd.DataFrame(columns=(“Time”, “Current”, “stdev”, “mean”, “max”, “min”,
      “Current (mc)”, “max (mc)”, “min (mc)”),
    index = [l.stem for l in files])
for i, file in enumerate(files):
  print(i, file)
  df1 = pd.read_csv(file)
  df1 = df1[abs(df1[“CH1 Current”]) < 1e30]
  df.iloc[i][“Time”] = df1[“CH1 Time”]
  df.iloc[i][“Current”] = df1[“CH1 Current”]
  df.iloc[i][“stdev”] = df1[“CH1 Current”].std( )
  df.iloc[i][“mean”] = df1[“CH1 Current”].mean( )
  df.iloc[i][“max”] = df1[“CH1 Current”].max( )
  df.iloc[i][“min”] = df1[“CH1 Current”].min( )
  #Calculates mean corrected values so the signal is centered around zero
  df.iloc[i][“Current (mc)”] = df.iloc[i][“Current”] − df.iloc[i][“mean”]
  df.iloc[i][“max (mc)”] = df.iloc[i][“Current (mc)”].max( )
  df.iloc[i][“min (mc)”] = df.iloc[i][“Current (mc)”].min( )
df[“max (mc)”] = pd.to_numeric(df[“max (mc)”])
df[“min (mc)”] = pd.to_numeric(df[“min (mc)”])
df[“stdev”] = pd.to_numeric(df[“stdev”])
#Water background max/min
# waterStdevMax = df[“stdev”][9:15].idxmax( )
# waterStdevMin = df[“stdev”][9:15].idxmin( )
# watermax = df.loc[waterStdevMax][“max (mc)”]
# watermin = df.loc[waterStdevMin][“min (mc)”]
#Water + micelles background max/min. Change the index values to grab the
#correct files corresponding to backgrounds
s1 = 1
e1 = 3
print(“backgrounds: “ + str(df.index.values[s1:e1]))
#Finds largest standard deviation of the backgrounds and takes the min/max
#from that measurement
micStdevMax = df[“stdev”][s1:e1].idxmax( )
micmax = df.loc[micStdevMax][“max (mc)”]
micmin = df.loc[micStdevMax][“min (mc)”]
#Water + micelles + analyte with background subtraction. Change index values
#to grab the correct files corresponding to analytes
s2 = 3
e2 = 6
print(“data: “ + str(df.index.values[s2:e2]))
#Set index range to the number of data points the electrometer takes.
Df2 = pd.DataFrame(columns=(df.index[s2:e2]), index = range (0,100000))
for name in df.index[s2:e2]:
  #Select all values where the value minus micmin is less than zero, i.e.
  #values negative enough that after background subtraction, there is still
  #signal since they are a negative value
  seriesmin = df.loc[name][“Current (mc)”][df.loc[name][“Current (mc)”]
        − micmin <0] micmin
  #Select all values where the value minus micmax is greater than zero. i.e.
  #values positive enough that after a background subtraction, there is
  #still signal since they are positive value
  seriesmax = df.loc[name][“Current (mc)”][df.loc[name][“Current (mc)”]
        − micmax >0] − micmax
  #Replace all null values (signal smaller than background) with zero
  df2[name] = seriesmax.add(abs(seriesmin), fill_value = 0)
  df2[name] = pd.to_numeric(df2[name])
df2 = df2.fillna(0)
df.to_csv(folder / “data.csv”)
df2.to_csv(folder / “analysis.csv”
```

### Listing 1

Any of the disclosed methods of data analysis can be implemented as computer-executable instructions stored on one or more computer-readable media (e.g., non-transitory computer-readable storage media, such as one or more optical media discs, volatile memory components (such as DRAM or SRAM), or nonvolatile memory components (such as hard drives and solid state drives (SSDs))) and executed on a computer (e.g., any commercially available computer, including smart phones or other mobile devices that include computing hardware such as processors that execute the computer-executable instructions). Tangible computer-readable storage media are any available tangible media that can be accessed within a computing environment (e.g., one or more optical media discs such as DVD or CD, volatile memory components (such as DRAM or SRAM), or nonvolatile memory components (such as flash memory or hard drives)). By way of example, computer-readable media include memory and storage. The terms computer-readable media and computer-readable storage media do not include transitory signals or carrier waves. In addition, the terms computer-readable media or computer-readable storage media do not include communication ports or transitory communication media.

Any of the computer-executable instructions for implementing the disclosed techniques, as well as any data created and used during implementation of the disclosed aspects, can be stored on one or more computer-readable media (e.g., non-transitory computer-readable storage media). The computer-executable instructions can be part of, for example, a dedicated software application, or a software application that is accessed or downloaded via a web browser or other software application (such as a remote computing application). Such software can be executed, for example, on a single local computer (e.g., implemented with general-purpose CPUs and/or specialized processors, such as graphics processing units (GPUs) or tensor processing units (TPUs); application-specific integrated circuits (ASICs), or programmable logic devices (PLDs), such as field programmable gate arrays (FPGAs) executing on any suitable commercially-available computer) or in a network environment (e.g., via the Internet, a wide-area network, a local-area network, a client-server network (such as a cloud computing network), or other such network) using one or more network computers. The integrated circuit or specialized computing hardware can be embedded in or directly coupled to a sensor in accordance with those described above.

For clarity, only certain selected aspects of the software-based implementations are described. Other details that are well known in the art are omitted. For example, it should be understood that the disclosed technology is not limited to any specific computer language or program. For instance, the disclosed technology can be implemented by software written in C, C++, C#, Forth, Fortran, Java, JavaScript, Lisp, MATLAB, Perl, Python, Ruby, or any other suitable programming language. Likewise, the disclosed technology is not limited to any particular computer or type of hardware.

Furthermore, any of the software-based aspects (comprising, for example, computer-executable instructions for causing a computer to perform any of the disclosed methods) can be uploaded, downloaded, or remotely accessed through a suitable communication means. Such suitable communication means include, for example, the Internet, the World Wide Web, an intranet, software applications, cable (including fiber optic cable), magnetic communications, electromagnetic communications (including RF, microwave, and infrared communications), electronic communications, or other such communication means.

The raw current and time data were parsed from the electrode and corrected to center around zero. From the noisiest background, taken from the determining the largest standard deviation, the maximum and minimum signals were taken. From the sample count data, the region between the maximum and minimum was removed. The resulting absolute value of the data was then plotted.

### Example 2: Air Sampling Compatible Detection of SARS-CoV-2 by Functionally Imprinted Micelles

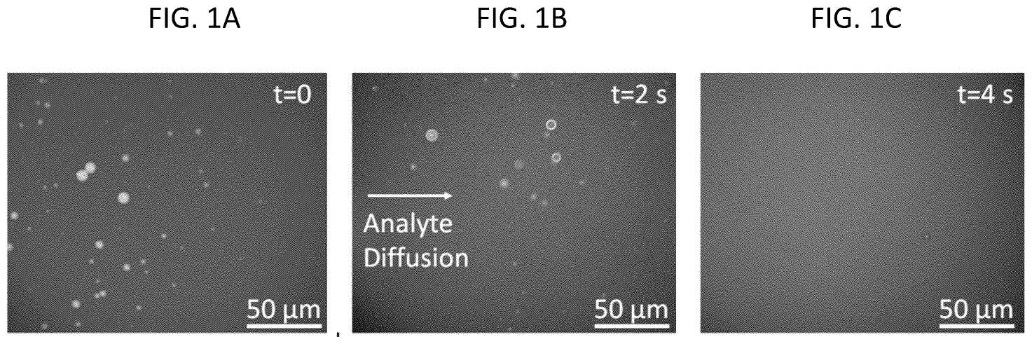

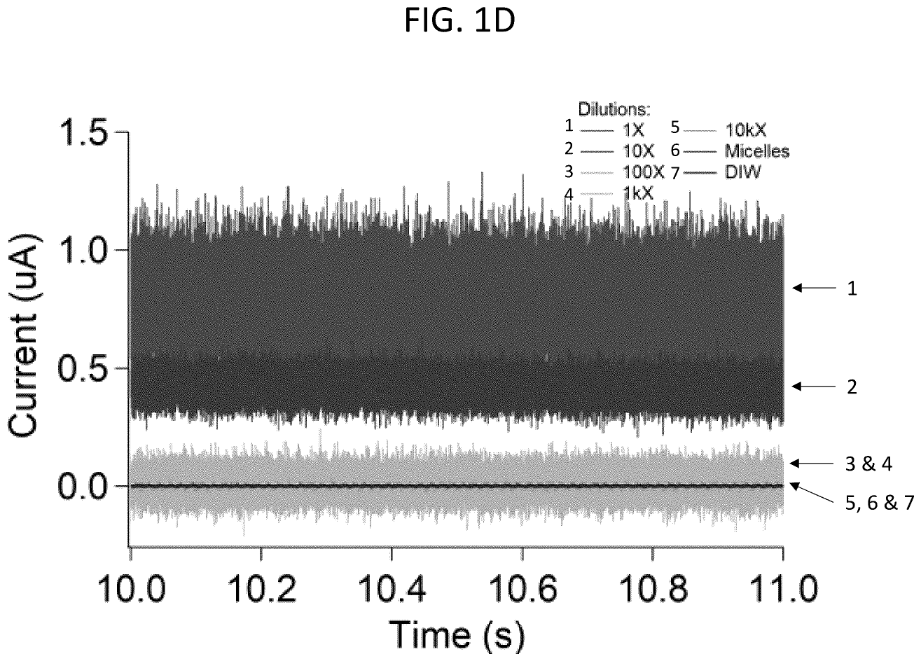

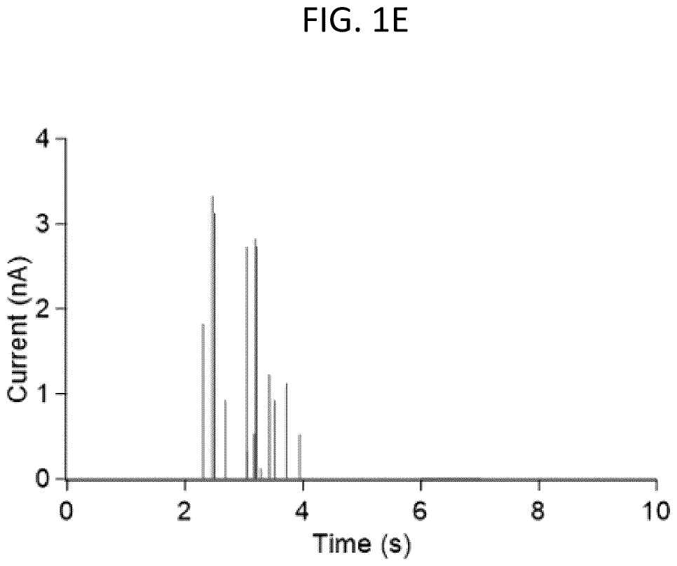

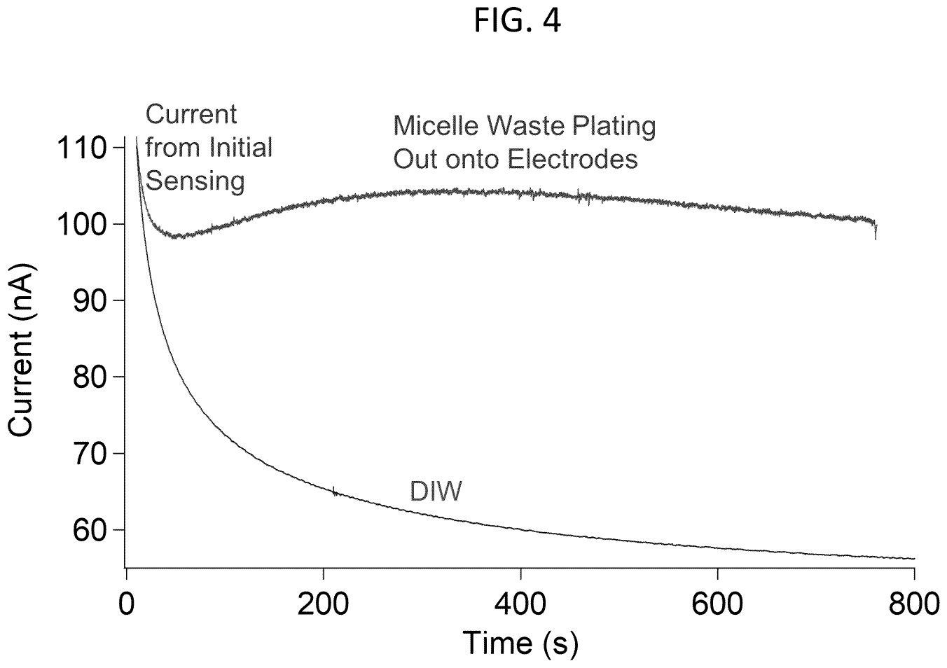

FIGS. 1A-1E demonstrate the imprinted micelle-analyte interaction optically and electronically. FIGS. 1A-1C show the imprinted micelles frame-by-frame as the analyte diffuses from left to right in the photoluminescent video stills. The micelles are seen to “pop” as they lose their luminescent midlayer (the linseed oil). As micellular ions are released into the bath, the minute changes in resistivity are registered as an electronic signal, which is exemplified in FIG. 1D and for longer time scales in FIG. 4. The long interaction time between the imprinted micelles and the analytes (organic sensitized silica) was investigated by addition of a 1 mg/mL suspension of analyte particles, in this case functionalized silica, into the electrochemical cell (FIG. 4).

FIG. 1D shows the electronic signals resulting from serial dilutions of analyte particles. A suspension of 2.5 ng of analyte particles (1× in the image legend, 5 μL of a 500 ng/mL suspension) was injected and evaluated. Subsequent dilutions of 10× were also evaluated for their electronic response. The results are visible in the graph until the 10 kX dilution was evaluated (250 fg). Subsequent data analysis resulted in isolated single micelle-analyte reactions being recorded by further diluted samples. FIG. 1E gives an overview of the results from five trials at sub-fg amounts of analyte (between 50 and 500 viruses/injection). As can be seen in FIG. 1E, after an initial mixing period, the system returned about 25 discrete sensitized micelle (SM)-analyte events.

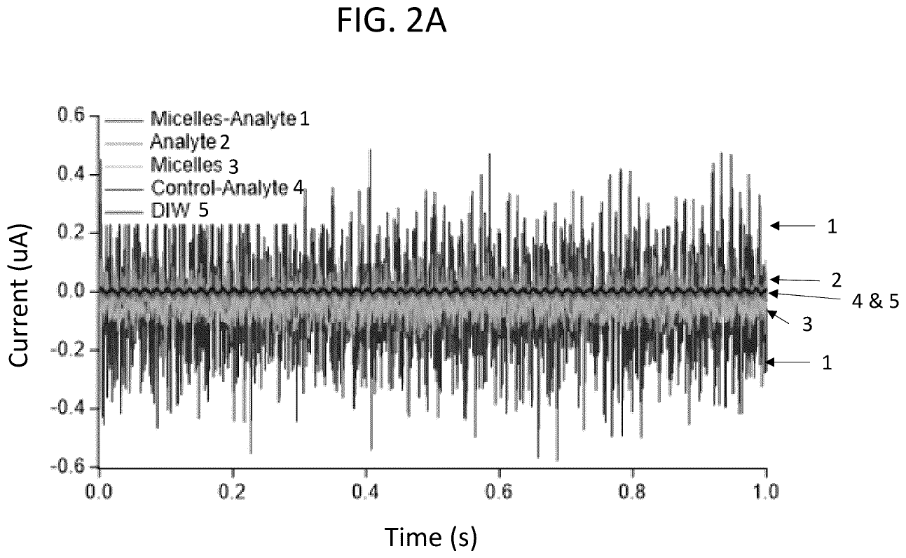

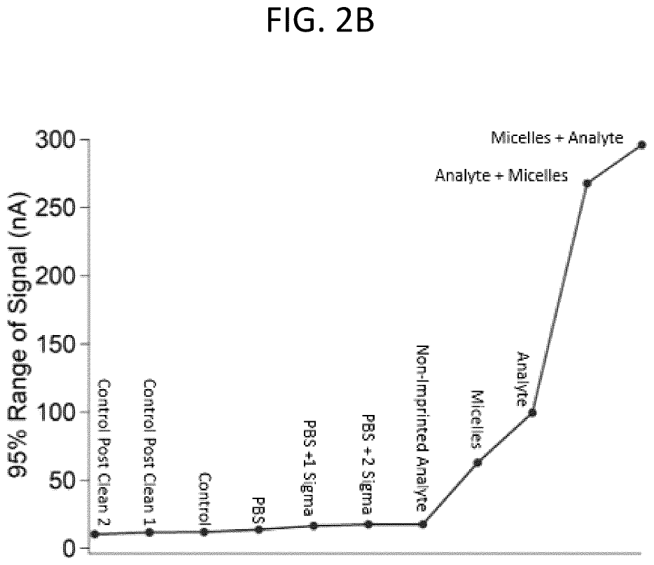

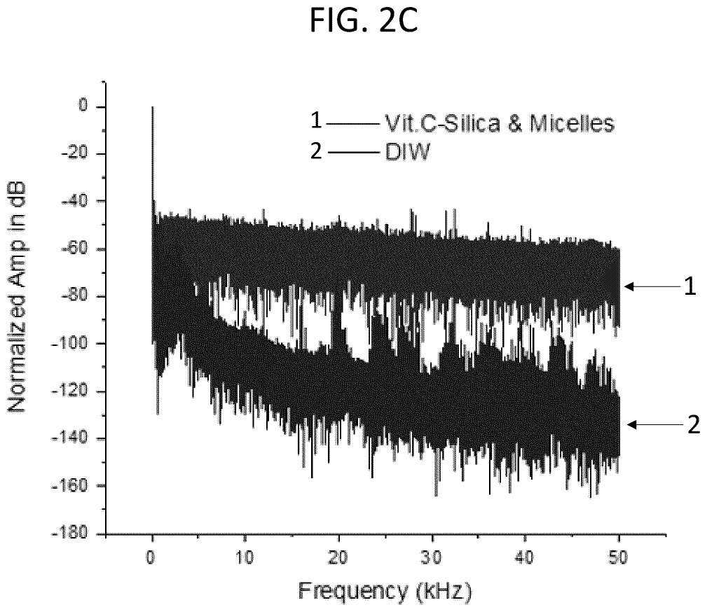

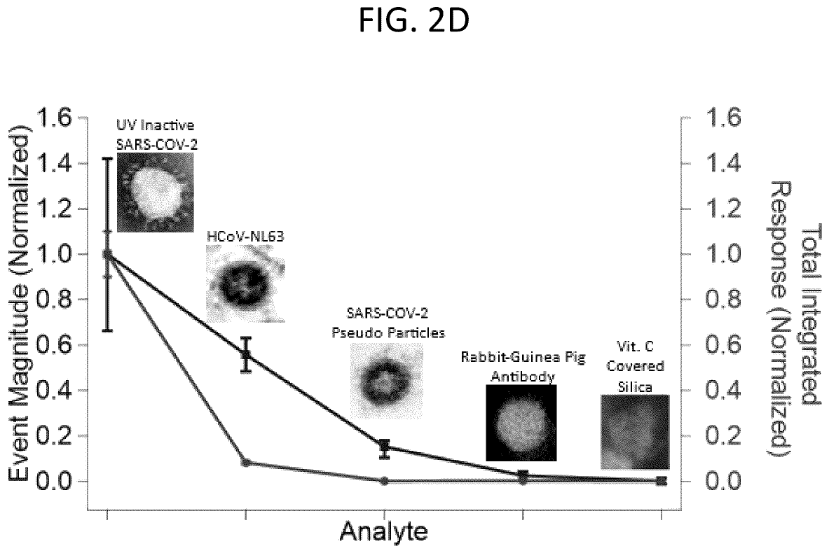

With a high degree of sensitivity established, the electronic response relative to several controls was evaluated to ensure that the measured signal originated from SM-analyte interaction (FIGS. 2A-2B). As seen in FIG. 2B, the imprinted micelle-analyte interactions produce significantly more signal than control measures. To ensure that the observed signals were not generated by noise on the power supply line, the frequency distribution was established. As shown in FIG. 2C, there is a stochastic aspect to the signal as expected from Brownian motion of diffusion.

As the sensitivity and SM-analyte interaction aspects of the signal were established, the selectivity of the SMs was then evaluated. FIG. 2D shows the normalized response and signal width (error bars, blue line) of SARS-CoV-2 imprinted micelles tested against other analytes. The total integrated response of each analyte is also presented. As can be seen in FIG. 2D, the signal is diminished significantly when other analytes are tested. Most notably, the SM system shows a difference between pseudotyped particles of SARS-CoV-2, active virus HCoV-NL63, and UV-inactivated SARS-CoV-2. While HCoV-NL63 did, in some instances, produce event magnitudes in line with the size of the SM-SARS-CoV-2 interactions, the total integrated response was very diminished.

As the sensitivity and selectivity of the SM system was established, an airborne particle collection system was designed that can integrate with the SM electrochemical cell. Initial airborne detection of analyte particles was accomplished, and an overview is presented in FIGS. 3A-3D. FIG. 3A is a macro image showing vortex mixing of the sampled ambient air containing analyte particles and mixing with a generated aqueous aerosol. In this manner, the particles from the air are transitioned from suspension in air to suspension in water particles (Cox et al., Aerosol Sci. Technol. 5:572-584, 2020; Liu et al., Nature 582:557-560, 2020; Riemer et al., Rev. Geophys. 57(2):187-249, 2019). FIG. 3B depicts the flow of the aerosol as it is condensed into a single liquid water volume and then stored at 0° C. (prior to testing in the electrochemical cell). FIG. 3C shows the high-resolution data of the cell response with an aliquot of the analyte particles that had been condensed into the liquid water. Lastly, FIG. 3E shows a longer time measurement to demonstrate how long analyte particles can generate a detectable response when injected into the SM cell.

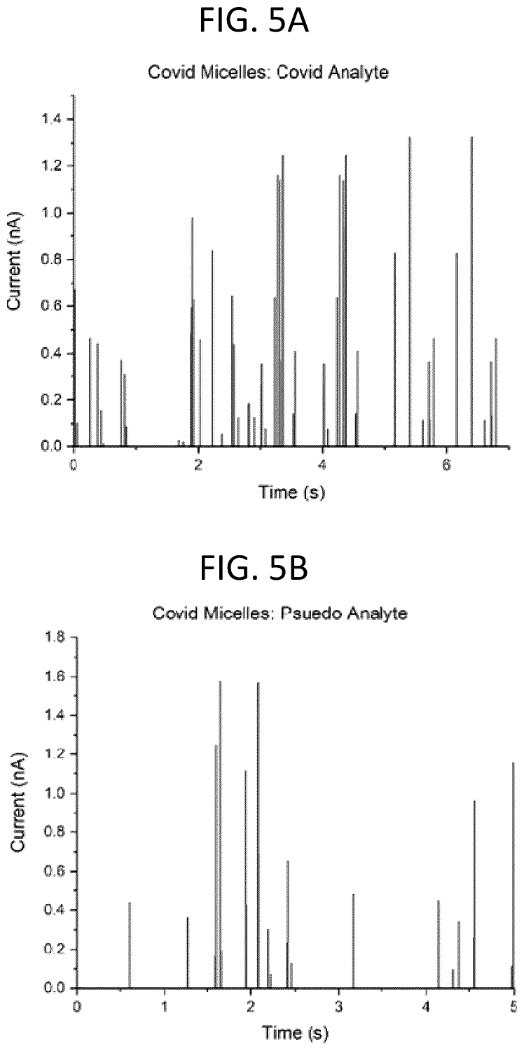

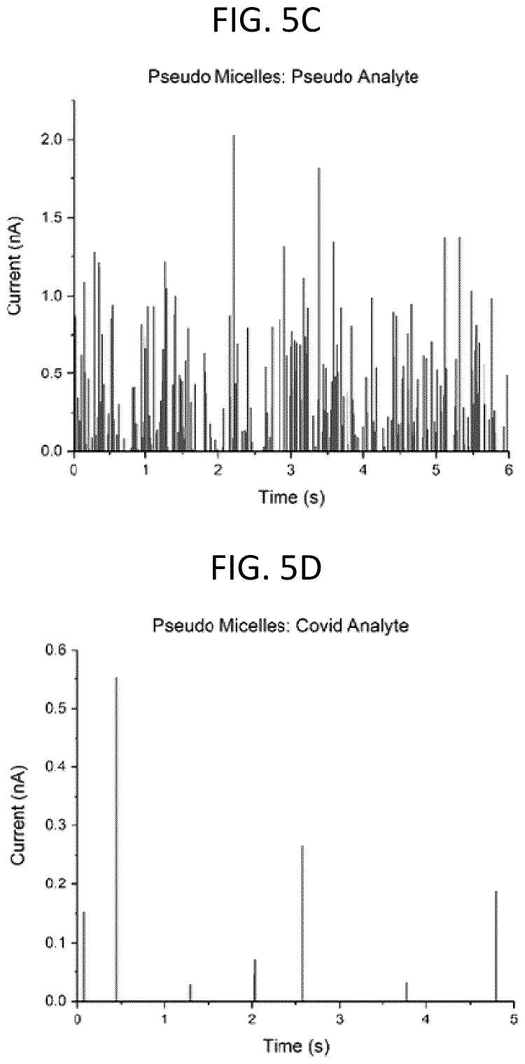

Next, the relative response of imprinted micelles sensitized to either UV-inactivated SARS-CoV-2 or pseudotyped particles of SARS-CoV-2 was determined. The response was measured via the electrochemical cell setup. 100 μl of DIW (cleaned at 2V for 48 hours) was injected into the vial and held at the analysis voltage for 15 minutes to remove any remaining electrolytes in the bath. When the measurements were obtained, 5 μL of the imprinted micelle solution was injected into the cleaned water, as well as 5 μL of the analyte corresponding to the micelle's sensitivity, then another 5 μL of the opposite analyte with measurements made after each addition. The UV-inactivated SARS-CoV-2 sensitized micelle response to UV-inactivated SARS-CoV-2 is shown in FIG. 5A. The addition of pseudotyped particles of SARS-CoV-2 created an additional response in FIG. 5B. The pseudotyped particles of SARS-CoV-2 sensitized micelle to the pseudotyped particles of SARS-CoV-2, as shown in FIG. 5C, responded stronger than in FIG. 5A. The addition of UV-inactivated SARS-CoV-2 produced little response, as shown in FIG. 5D.

These results demonstrate that the electronic detection of picomolar levels of analyte into a sub-mL bath is supported by the results of FIGS. 1A-1E. The sensitivity of the system and SM-analyte interaction at the pg/mL levels of analyte show that the imprinted micelle technology can generate signals from picomolar levels and perhaps smaller. The proposal that the functionalization of micelles with imprinted particles is supported by the many backgrounds and control evaluations in FIGS. 2A-2D. The imprinted micelles responded to the presence of imprinted analytes with a degree of specificity, resulting in a larger electronic signal than background evaluation or the presence of particles not imprinted on the micelle. As demonstrated by the results in FIGS. 3A-3D, the electrochemical SM cell is compatible with biological aerosol sampling methods. The combination of the cell results indicate that it is possible to integrate the use of imprinted SMs, commercial electronics, and established bioaerosol sampling methods to real-time field-based detection of analyte particles of interest.

Electronic signals have been generated from functionalized micelles, imprinted to specific analytes of interest. The ability to detect analytes of interest, such as SARS-CoV-2, with a system that can be adapted for field use allows for wide-scale deployment of imprinting technology. The technology can be used for industrial pollutant detection, vial emissions in buildings, the presence and growth of bacterial species in foods, and many other applications involving the detection of compounds in an ambient setting.

It will be apparent that the precise details of the methods or compositions described may be varied or modified without departing from the spirit of the described aspects of the disclosure. We claim all such modifications and variations that fall within the scope and spirit of the claims below.

1. A method of detecting an airborne analyte, comprising:
contacting an ambient air sample suspected of containing the analyte with an aqueous aerosol and subjecting the air sample and aqueous aerosol to condensation, thereby producing a liquid-analyte solution;
contacting the liquid-analyte solution with an imprinted micelle, wherein the imprinted micelle comprises an analyte imprint and contains a salt or electrolyte solution; and
detecting an electrochemical signal produced upon binding of the analyte to the imprinted micelle, releasing the salt solution, thereby detecting the airborne analyte.

2. The method of claim 1, wherein the analyte comprises a pathogen, a toxin, a protein, an organic molecule, an inorganic particle, a chemical, an explosive particle, or an environmental pollutant.

3. The method of claim 2, wherein the pathogen is a virus, a bacterium or a fungus.

4. The method of claim 3, wherein the virus is a human coronavirus.

5. The method of claim 4, wherein the human coronavirus is a severe acute respiratory syndrome coronavirus 2 (SARS-CoV-2), SARS-CoV, Middle East respiratory syndrome coronavirus (MERS-CoV), human coronavirus HKU1 (HKU1-CoV), human coronavirus OC43 (OC43-CoV), human coronavirus 229E (229E-CoV), or human coronavirus NL63 (NL63-CoV).

6. The method of claim 1, wherein the imprinted micelle is a double-layered micelle, wherein the outer layer of the micelle comprises the analyte imprint, and the analyte imprint comprises at least one molecularly imprinted particle specific for the analyte.

7. The method of claim 6, wherein the at least one molecularly imprinted particle comprises silica nanoparticles, magnetic nanoparticles, gold nanoparticles, polymer nanoparticles, polymer-silica composite nanoparticles, quantum dots, or nanoclusters.

8. The method of claim 6, wherein the at least one molecularly imprinted particle comprises 5 to 10 molecularly imprinted particles per micelle.

9. The method of claim 1, wherein the liquid-analyte solution and imprinted micelle are contacted in an electrochemical cell.

10. The method of claim 9, wherein the electrochemical cell comprises a glass vessel containing deionized water.

11. The method of claim 10, wherein the glass vessel is coated with polystyrene, polytetrafluoroethylene (PTFE), alumina, silica or a high-κ dielectric material.

12. The method of claim 9, wherein the imprinted micelle is adhered to a solid support in the electrochemical cell.

13. The method of claim 12, wherein the solid support is coupled to a conductive fiber filter.

14. The method of claim 1, wherein the salt solution comprises ferrous cyanide, sodium chloride, or fluorescein.

15. The method of claim 1, wherein the method is a multiplex method, and the imprinted micelle comprises imprinted micelles specific for a plurality of analytes.

16. The method of claim 15, wherein each imprinted micelle comprises at least one molecularly imprinted particle specific for each analyte.

17. The method of claim 15, wherein each imprinted micelle is specific for one analyte.

18. The method of claim 15, wherein the plurality of analytes comprises at least two, at least three, at least four, at least four or at least five different viruses.

19. The method of claim 18, wherein the viruses are respiratory viruses.

20. The method of claim 19, wherein the respiratory viruses are selected from a coronavirus, a respiratory syncytial virus, an influenza virus, a rhinovirus, an adenovirus and a parainfluenza virus.

21. The method of claim 1, wherein the sample comprises no more than 50, no more than 25, no more than 10, or no more than 5 particles of the analyte.

22. The method of claim 1, further comprising, with a processor:
receiving data from an electrometer coupled to a sensor comprising the imprinted micelle; and
comparing the data to background data to estimate particle-micelle interactions.

23. Computer-readable storage media storing computer-executable instructions, which when executed by the processor, cause a computer to perform the method of claim 22.

24. An apparatus comprising:
memory; and
the processor configured to execute the computer-executable instructions stored on the computer-readable storage media of claim 23.
